(19) United States
(12) Patent Application Publication
(10) Pub. No.: US 2023/0280423 A1
(43) Pub. Date: Sep. 7, 2023

(54) MAGNETIC FIELD GENERATION DEVICE AND MAGNETIC FIELD IRRADIATION METHOD

(71) Applicant: National University Corporation Tokai National Higher Education and Research System, Chikusa-ku, Nagoya-shi, Aichi (JP)

(72) Inventors: Kinji OHNO, Nagoya-shi, Aichi (JP); Mikako ITO, Nagoya-shi, Aichi (JP); Takuro TODA, Nagoya-shi, Aichi (JP)

(21) Appl. No.: 18/005,664

(22) PCT Filed: Jun. 2, 2021
(86) PCT No.: PCT/JP2021/020930

§ 371 Date: Jan. 16, 2023

(30) Foreign Application Priority Data
Jul. 16, 2020 (JP) 2020-122378

(51) Int. Cl. G01R 33/36

(52) U.S. Cl. CPC G01R 33/3607

(57) ABSTRACT

A problem is to provide a magnetic field generation device and magnetic field irradiation method that are useful to a living body. This magnetic field generation device includes a coil and a power source. The problem can be solved by the magnetic field generation device of which the power source can apply, to the coil, an electric current that is pulsed and that has frequency fluctuation, a maximum value of a generated magnetic field being 60 mG to 3000 mG.

### TECHNICAL FIELD

The disclosure in the present application relates to a magnetic field generation device and a magnetic field irradiation method.

### BACKGROUND ART

It is known that it is possible to treat various diseases by irradiating a living body, for example, a human body or the like with a magnetic field.

For example, a cancer treatment device that suppresses cancer cell growth by applying an alternating magnetic field at any frequency from 100 kHz to 300 kHz to an affected tissue is known (see Patent Literature 1). Further, it is known that a blood flow increases when a ferrite magnet generating a weak magnetic field of 0.3 Gauss or higher and 0.5 Gauss or lower is attached to a patient (see Patent Literature 2).

### CITATION LIST

### Patent Literature

Patent Literature 1: Japanese Patent No. 6603812

Patent Literature 2: Japanese Patent Application Laid-Open No. 2016-93229

### SUMMARY OF INVENTION

### Technical Problem

As is clear from the disclosure in Patent Literature 1 and Patent Literature 2 described above, in treatment of a disease using a magnetic field, the conditions of the irradiating magnetic field (intensity, frequency, or the like) will differ in accordance with a targeted disease. Thus, the present inventors have made an intensive study about the relationship between conditions of a magnetic field and diseases and newly found that:
(1) an extremely weak magnetic field such that the maximum value of a generated magnetic field is 60 mG to 3000 mG; and
(2) rather than using a constant frequency, modulating the frequency of pulsed current applied to a coil used for generating a magnetic field
   are useful for a living body.

That is, an object of the disclosure in the present application is to provide a magnetic field generation device and a magnetic field irradiation method that are useful for a living body.

### Solution to Problem

The disclosure of the present application relates to a magnetic field generation device and a magnetic field irradiation method illustrated below.
(1) A magnetic field generation device comprising:
   a coil; and
   a power supply,
   wherein the power supply is configured to apply pulsed and frequency-modulated current to the coil, and
   wherein the maximum value of a generated magnetic field is 60 mG to 3000 mG.
(2) The magnetic field generation device according to (1) above, wherein a pulse width of the current is selected from 2 to 8 msec.
(3) The magnetic field generation device according to (1) or (2) above, wherein the power supply is configured to repeatedly apply
   a cycle in which the frequency increases during a predetermined period, or
   a cycle in which the frequency decreases during a predetermined period
      to the coil.
(4) The magnetic field generation device according to (3) above,
   wherein the frequency is the number of pulses applied to the coil per second, and
   wherein during the predetermined period,
   the frequency increases stepwise within a range selected from 1 Hz to 8 Hz, or
   the frequency decreases stepwise within a range selected from 8 Hz to 1 Hz.
(5) The magnetic field generation device according to (3) or (4) above, wherein the predetermined period is selected from 2 sec to 8 sec.
(6) The magnetic field generation device according to any one of (1) to (5) above, wherein the magnetic field generation device is used for treatment of a mitochondria-related disease.
(7) A magnetic field irradiation method for a living body excluding a human body by using a magnetic field generation device including a coil and a power supply, the magnetic field irradiation method comprising:
   a magnetic field irradiation step of irradiating a living body with a magnetic field having the maximum value of 60 mG to 3000 mG generated by the magnetic field generation device,
   wherein in the magnetic field irradiation step, the power supply applies pulsed and frequency-modulated current to the coil.
(8) The magnetic field irradiation method according to
(7) above, wherein a pulse width of the current is selected from 2 to 8 msec.
(9) The magnetic field irradiation method according to (7) or (8) above, wherein the power supply is configured to repeatedly apply
   a cycle in which the frequency increases during a predetermined period, or
   a cycle in which the frequency decreases during a predetermined period
      to the coil.
(10) The magnetic field irradiation method according to (9) above,
   wherein the frequency is the number of pulses applied to the coil per second, and
   wherein during the predetermined period,
   the frequency increases stepwise within a range selected from 1 Hz to 8 Hz, or
   the frequency decreases stepwise within a range selected from 8 Hz to 1 Hz.
(11) The magnetic field irradiation method according to (9) or (10) above, wherein the predetermined period is selected from 2 sec to 8 sec.
(12) The magnetic field irradiation method according to any one of (7) to (11) above, wherein the magnetic field irradiation method is used for a method of treating a mitochondria-related disease.

### Advantageous Effect

The magnetic field generation device and the magnetic field irradiation method disclosed in the present application are useful for a living body.

### BRIEF DESCRIPTION OF DRAWINGS

FIG. 1A and FIG. 1B are schematic diagrams illustrating an example of an embodiment of a magnetic field generation device.

FIG. 2 is a diagram illustrating pulsed current and frequencies (Hz).

FIG. 3 is a diagram illustrating an overview when frequency-modulated current is applied.

FIG. 4 is a photograph substitute for a drawing, which is a photograph of a magnetic field generation device fabricated in Example 1.

FIG. 5A is a photograph substitute for a drawing, which is a photograph representing arrangement of a coil and a petri dish of a magnetic field generation device in Example 2. FIG. 5B is a graph illustrating the decreased mitochondrial mass per cell after AML12 cells were irradiated with a magnetic field for 3 hours.

FIG. 6 is a graph illustrating the decreased mitochondrial mass per cell after AML12 cells were irradiated with a magnetic field for 12 hours.

FIG. 7 is a graph illustrating the increased mitochondrial membrane potential per cell after AML12 cells were irradiated with a magnetic field for 12 hours.

FIG. 8A is a graph illustrating a profile when a Mito stress kit was used. FIG. 8B is a graph illustrating changes in the amount of the oxygen consumption when NARP3-2 cybrids were irradiated with a magnetic field and when NARP3-2 cybrids were not irradiated with a magnetic field. FIG. 8C is a graph illustrating changes in the amount of the oxygen consumption when NARP3-1 cybrids were irradiated with a magnetic field and when NARP3-1 cybrids were not irradiated with a magnetic field.

FIG. 9 is a graph illustrating the decreased mitochondrial mass of AML12 cells when current at different frequencies was applied to the coil.

FIG. 10 is a graph illustrating the decreased mitochondrial mass of AML12 cells when current with different pulse widths was applied to the coil.

FIG. 11 is a graph illustrating the decreased mitochondrial mass when different types of cells were irradiated with a magnetic field.

FIG. 12A is a graph illustrating a result of a Rota rod test when Parkinson's disease model mice were irradiated with a magnetic field. FIG. 12B is a graph illustrating a result of an inverted grid hanging test when Parkinson's disease model mice were irradiated with a magnetic field.

FIG. 13A is a diagram illustrating a method of creating a depression model mouse. FIG. 13B is a diagram illustrating an experiment procedure of a swimming test of the depression model mouse.

FIG. 14 is a graph illustrating a result of a swimming test when depression model mice were irradiated with a magnetic field.

### DETAILED DESCRIPTION OF EMBODIMENTS

A magnetic field generation device and a magnetic field irradiation method disclosed in the present application will be described below in detail. Note that some of the position, the size, the range, or the like of respective components illustrated in the drawings do not represent the actual position, the actual size, the actual range, or the like for easier understanding. Thus, the disclosure of the present application is not necessarily limited to the position, the size, the range, or the like disclosed in the drawings.

Further, in the present specification, it is construed that:
(1) a numeric range represented by using “to” means a range including the numerical values stated before and after “to” as the lower limit value and the upper limit value;
(2) a numerical value, a numeric range, and a qualitative expression (for example, an expression of “identical”, “the same”, or the like) represents a numerical value, a numeric range, and a nature including an error tolerated in general in this technical field; and
(3) reference to “substantially XX-shape(d)” includes a shape recognized as approximately the XX-shape(d) in addition to an accurate XX-shape(d).

### Embodiment of Magnetic Field Generation Device

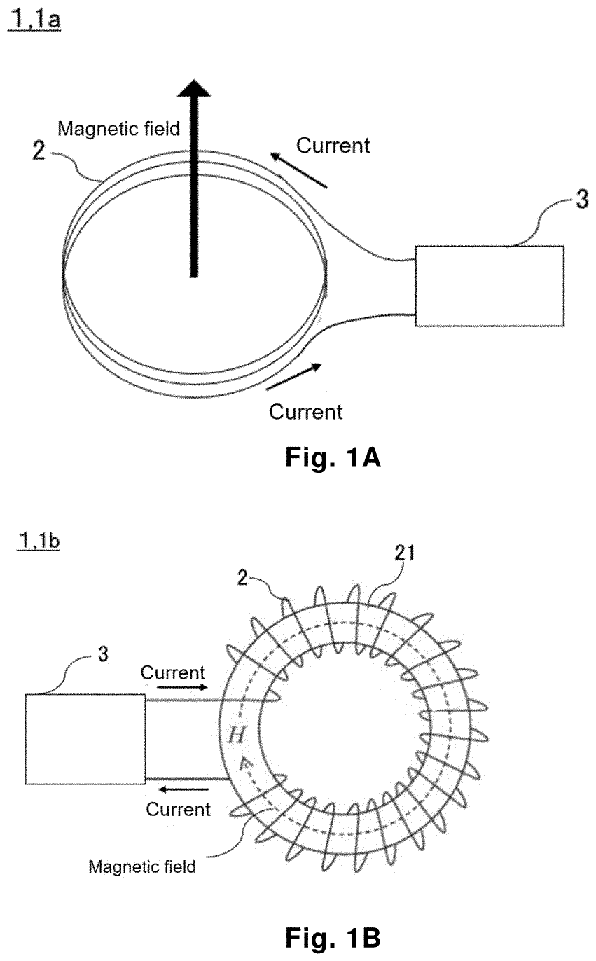

An embodiment of a magnetic field generation device 1 will be described with reference to FIG. 1. FIG. 1A and FIG. 1B are schematic diagrams illustrating an example of the embodiment of the magnetic field generation device.

A magnetic field generation device la according to the embodiment illustrated in FIG. 1A includes a coil 2 and a power supply 3.

The coil 2 is not particularly limited as long as it can generate a magnetic field when current is supplied from the power supply 3. The material forming the coil 2 may be any material as long as it is an electroconductive material and may be, for example, an electroconductive metal such as silver, copper, gold, aluminum, zinc, iron, tin, lead, or the like or an alloy containing the electroconductive metal. Further, the coil 2 can be fabricated by winding a wire material made of the material described above, and the wire material may be a single wire or may be a litz wire.

A generated magnetic field will be stronger when:
(1) the number of turns per unit length of the coil 2 is larger;
(2) the diameter of the wire material forming the coil 2 is larger; and
(3) the value of current applied to the coil 2 is larger.
   Therefore, the number of turns of the coil 2 or the diameter of the wire material can be adjusted as appropriate together with the value of current so that a magnetic field intensity described later can be obtained.

FIG. 1A illustrates the coil 2 fabricated by winding a wire material on a cylinder and then pulling out the cylinder. Alternatively, although illustration is omitted, the coil 2 may be wound on a support such as a cylinder. Further, although the coil 2 is formed by winding a wire material in the example illustrated in FIG. 1A, alternatively, the coil 2 may be formed by printing a pattern on a printed board such as an FPC.

FIG. 1B illustrates an example in which a wire material is wound spirally on an annular support 21 having a hollow inside and thereby the coil 2 is fabricated. In the example illustrated in FIG. 1B, substantially a circular magnetic field H passing through substantially the center of the annular support 21 is generated. Note that, although FIG. 1B illustrates the example with the annular support 21, the support 21 may be omitted when the rigidity of the wire material is high.

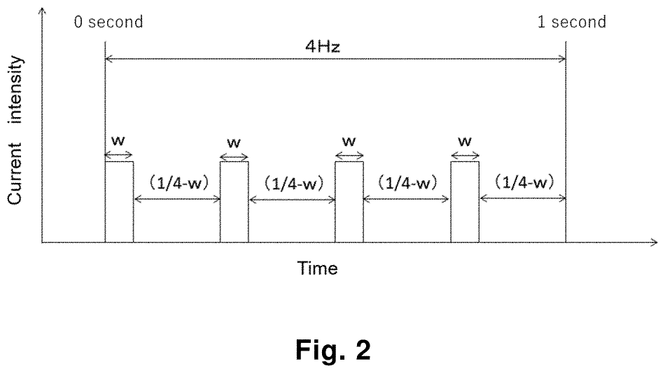

The power supply 3 is not particularly limited as long as it can apply pulsed and frequency-modulated current to a coil. First, the pulsed current and the frequency (Hz) will be described with reference to FIG. 2. The pulsed current applied by the power supply 3 means current having substantially a rectangular waveform whose pulse width (application duration) is w sec (second). Further, in the present specification, reference to “frequency” means the number of repetitions of [“application of pulsed current having a pulse width w (application duration w sec)”+“interval time of applied current of 0 A”] per second. FIG. 2 illustrates an example in which the frequency is 4 Hz, a unit of “application of pulsed current having a pulse width w (application duration w sec) and then an interval of applying current of 0 A ((¼−w) sec)” is repeated for four times. That is, in the present specification, reference to a frequency of x Hz means that a unit of [“application of pulsed current having a pulse width w (application duration w sec)” and then “interval time of applying current of 0 A ((1/x−w) sec)”] is repeated for x times.

The pulse width is not particularly limited as long as a generated magnetic field is useful for a living body. The pulse width may be, for example, 1.5 msec to 12 msec, preferably, 2 msec to 8 msec. The frequency is also not particularly limited as long as a generated magnetic field is useful for a living body. The frequency may be, for example, 1 Hz to 12 Hz, preferably, 1 Hz to 8 Hz.

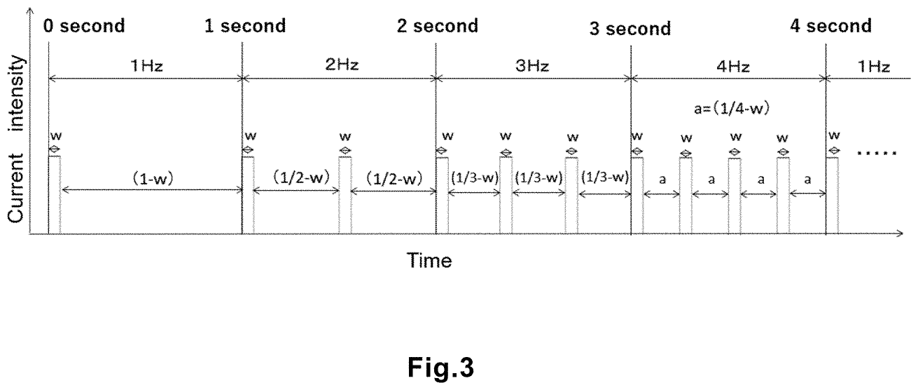

FIG. 3 is a diagram illustrating an overview when frequency-modulated current is applied. FIG. 3 illustrates an example in which current whose frequency is modulated stepwise at 1 Hz, 2 Hz, 3 Hz, and 4 Hz in this order is applied to a coil. In the example illustrated in FIG. 3, application of current whose frequency increases stepwise such as “1 Hz to 2 Hz to 3 Hz to 4 Hz” is defined as one cycle, and the cycle of “1 Hz to 2 Hz to 3 Hz to 4 Hz” is then repeatedly applied to the coil. Note that, in the present specification, the period for implementing one cycle may be referred to as a “predetermined period”.

The magnetic field generation device 1 according to the embodiment is not particularly limited as long as it modulates the frequency of current applied from the power supply 3 to the coil 2 during a predetermined period (one cycle). FIG. 3 illustrates an example in which the frequency is increased stepwise in one cycle. Alternatively, the frequency may be reduced stepwise during a predetermined period (one cycle), or otherwise an increase and a reduction of the frequency may be combined. As illustrated in Examples and Comparative examples described later, application of frequency-modulated current to the coil achieves a useful effect on the living body. The frequency may be selected as appropriate between the upper limit and the lower limit defined by the frequencies described above as an example. The predetermined period is not particularly limited as long as a generated magnetic field is useful for a living body. The predetermined period may be, for example, 2 to 8 sec.

Even with the same predetermined period, a change of the number of modulated frequencies will cause a change of the duration of current application for each frequency. A duration of current application for an individual frequency in one cycle may be, for example, 1 sec to 2 sec. Further, the durations of current application for individual frequencies in one cycle may be the same as or different from each other. For example, in the example illustrated in FIG. 3, all the durations of current application for four different frequencies (1 Hz, 2 Hz, 3 Hz, 4 Hz) are each 1 sec in one cycle (the predetermined period of 4 sec). Alternatively, the duration of application may be changed in accordance with a frequency such as 0.5 sec for 1 Hz and 2 Hz, 1.5 sec for 3 Hz and 4 Hz, or the like, for example.

The maximum value of a generated magnetic field is not particularly limited as long as it is useful for a living body. The maximum value may be, for example, 60 mG to 3000 mG, more preferably, 100 mG to 3000 mG. Note that, in the present specification, the maximum value of a magnetic field means an actual measured value and/or a theoretical value of a generated magnetic field. The theoretical value may be calculated from the material forming the coil 2, the size and the number of turns of the coil, the value of current, or the like (calculated theoretical value). Further, the magnetic field intensity generated when a predetermined value of current is applied by using a fabricated magnetic field generation device is measured, and a theoretical value (theoretical equation) may be created based on the actual measured value. Alternatively, a theoretical value (actual measured-calculated theoretical equation) may be created taking a difference or the like between a calculated theoretical value and an actual measured value into consideration. In the example illustrated in FIG. 1A, since the coil 2 is substantially a circular shape, the strongest magnetic field occurs at the center of substantially the circular shape. Further, in the example illustrated in FIG. 1B, the strongest magnetic field occurs at the center of the cross section of substantially the annular support 21 (the dotted line in FIG. 1B). When a magnetic field is actually measured, a known magnetic field measuring device can be used for the measurement.

Although differing in accordance with a location, it is said that the earth's geomagnetic field has an intensity of about 500 mG in a mid-latitude region. The magnetic field generation device 1 disclosed in the present application generates an extremely weak magnetic field that is substantially the same as the geomagnetic field. It has been newly found by the present inventors that a useful effect on a living body is achieved by using the weak magnetic field and further changing the frequency of current applied to the coil 2 (in other words, changing the frequency of a magnetic field).

As illustrated in Examples and Comparative examples described later, it has been confirmed that the magnetic field generation device 1 according to the embodiment irradiates a cell, this first induces mitophagy of mitochondria, and the mitochondria are then activated. Further, in an experiment using Parkinson's disease model mice and depression model mice, improvement of the symptom thereof was found.

The mitophagy is a system in which:
(1) via PINK1 (encoding kinase) and Parkin (encoding ubiquitin ligase) known as a gene responsible for Parkinson's disease and a protein such as LC3,
(2) damaged abnormal mitochondria are selectively removed (degraded), and
(3) a path related to mitochondrial renewal is then promoted, and new mitochondria of good quality are produced. The mitophagy is known as a system intended mainly to maintain the quality of mitochondria.

Further, it is known that dysfunction of mitophagy relates to diseases such as mitochondrial diseases, neurodegenerative diseases, cardiac diseases, and the like (Um and Yun, “Emerging role of mitophagy in human diseases and physiology”, BMB Rep., 2017; 50(6): 299-307). Therefore, the magnetic field generation device disclosed in the present application achieves a therapeutic, palliative, or preventive effect on diseases or disorder due to dysfunction of mitophagy or accumulation of abnormal mitochondria.

Diseases on which use of the magnetic field generation device disclosed in the present application has an effect are illustrated as examples in the following (I) to (III). Note that a disease considered to be caused by abnormality of mitochondria described in (I) to (III) may be referred to as “mitochondria-related disease”. The following mitochondria-related diseases are mere examples, and the disclosure is not limited thereto.

### (I) Mitochondrial Disease (Energy Production Disorder Due to Accumulation of Abnormal Mitochondria is Considered to be a Cause)

Chronic progressive external ophthalmoplegia (CPEO); mitochondrial encephalomyopathy, lactic acidosis, stroke-like attack syndrome (MELAS); syndrome, myoclonus epilepsy associated with ragged-red fivers (MERRF); Leigh's encephalopathy (Leigh's syndrome); neurogenic muscle weakness, ataxia, and retinitis pigmentosa (NARP); Leber's hereditary optic neuropathy; Kearns-Sayre Syndrome (KSS); mitochondrial recessive ataxia syndrome (MIRAS); Mohr-Tranebjaerg syndrome; Bjornstad syndrome; multiple Mitochondrial Dysfunction Syndrome (MMDS); mitochondrial DNA depletion syndrome; mitochondrial diabetes; mitochondrial disease-related psychiatric disorders (Grainne S. et al., “Mitochondrial diseases”, Nat Rev Dis Primers, Vol. 2, No.16081, 2016).

### (II) Neurodegenerative Disease (Disorder of Quality Control Mechanism Mitophagy of Mitochondria is Considered to be One of Causes)

### (1) Parkinson's Disease

Since gene mutation of molecules of PINK1 and Parkin that are keys to mitophagy causes Parkinson's disease, disorder of mitophagy is also considered to occur in sporadic Parkinson's disease. In a behavioral experiment using ASO mice, a significant therapeutic effect on Parkinson's disease was found (Brent J. et al., “Mitochondrial Dysfunction and Mitophagy in Parkinson's: From Familial to Sporadic Disease”, Trends Biochem Sci., Vol. 40, No. 4, April 2015, P200-210).

### (2) Amyotrophic Lateral Sclerosis (ALS)

Since gene mutation of a molecule of optineurin (OPTN) that is a key to mitophagy causes ALS, disorder of mitophagy is also considered to occur in sporadic ALS (Wong Y. C., et al., “Optineurin is an autophagy receptor for damaged mitochondria in parkin-mediated mitophagy that is disrupted by an ALS-linked mutation”, Proc Natl Acad Sci USA, 2014; 111(42): E4439-48).

### (3) Huntington's Disease (HD)

Mitochondrial disorder is significantly responsible for the pathogenesis of HD (Khalil B. et al., “PINK1-induced mitophagy promotes neuroprotection in Huntington's disease”, Cell Death and Disease, (2015)6, e1617).

### (4) Alzheimer's Disease

Mitochondrial dysfunction and accumulation of damaged mitochondria are significantly responsible for the pathogenesis of AD (Fang E F. Mitophagy and NAD(+) inhibit Alzheimer disease. Autophagy 15: 1112-1114, 2019).

### (5) Depression

As with Examples described later, in a behavioral experiment using depression model mice subjected to a forced swimming test, a significant therapeutic effect on depression was found. Note that it is reported that depression is related to mitochondrial dysfunction (Husseini M. et al., “Impaired Mitochondrial Function in Psychiatric Disorders”, Nat Rev Neurosci, 2012 Apr. 18; 13(5): 293-307).

### (III) Ischemic Disease (Accumulation of Damaged Mitochondria Due to Incomplete Mitophagy Induces Insufficient Energy Production)

Ischemic heart disease, ischemic brain injury, ischemia-reperfusion injury, limb blood flow disorder (Buerger's disease, arteriosclerosis obliterans, and the like), respiratory dysfunction (Tang Y C. et al., “The critical roles of mitophagy in cerebral ischemia”, Protein Cell: 2016, 7(10): 699-713).

As described above, the magnetic field generation device disclosed in the present application is useful in particular for treatment or mitigation against diseases or disorder due to dysfunction of mitophagy or accumulation of abnormal mitochondria, however, the use thereof is not limited to disease treatment. Mitophagy is a function possessed by any organisms having mitochondria. Therefore, the magnetic field generation device disclosed in the present application is expected to achieve an effect of promoting mitophagy regardless of the presence or absence of a disease and thus is useful for a living body having mitochondria.

Mitochondria are organelles included in cells of eukaryotes. Therefore, a living body may be a eukaryote such as an animal, a plant, a fungus, a protist, or the like.

The usage method of the magnetic field generation device disclosed in the present application is not particularly limited as long as it can irradiate a living body with a generating magnetic field. For example, when cells or a small animal such as a mouse is irradiated with a magnetic field, a petri dish for culturing the cells or a cage for keeping the small animal can be arranged in a direction in which a magnetic field occurs (upstream of the coil in the example illustrated in FIG. 1A). Further, a plurality of coils 2 may be combined and used. For example, when irradiating a human body, by arranging a plurality of coils 2, one of which is illustrated in FIG. 1A, on a mat or the like so that a magnetic field occurs upward and allowing the human to lie on the mat, it is possible to irradiate the human body with a magnetic field during their sleeping. Further, the diameter of the coil 2 illustrated in FIG. 1A may be increased, and a living body such as a human body may be arranged inside the coil 2. For example, the coil 2 may be fabricated by winding a wire material around a bed. Alternatively, a living body may be arranged in a place where the magnetic field occurs inside the annular coil 2 illustrated in FIG. 1B.

### Embodiment of Magnetic Field Irradiation Method

Next, an embodiment of the magnetic field irradiation method will be described. Note that the magnetic field generation device used in the embodiment of the magnetic field irradiation method, more specifically, the coil, the power supply, the intensity of a generated magnetic field, the pulse width and the frequency of current applied to the coil, the definition of the frequency-modulated current, the predetermined period, and the definition of a living body or the like are the same as those in the embodiment of the magnetic field generation device. Therefore, in the embodiment of the magnetic field irradiation method, a magnetic field irradiation step will be mainly described, and duplicated description for the features that have already been described in the embodiment of the magnetic field generation device will be omitted. It is thus obvious that, even though not explicitly described in the embodiment of the magnetic field irradiation method, the features that have already been described in the embodiment of the magnetic field generation device can be employed in the embodiment of the magnetic field irradiation method.

The embodiment of the magnetic field irradiation method uses the magnetic field generation device including a coil and a power supply. Further, the magnetic field irradiation method includes a magnetic field irradiation step of irradiating a living body with a magnetic field having the maximum value of 60 mG to 3000 mG generated by the magnetic field generation device, and in the magnetic field irradiation step, the power supply applies pulsed and frequency-modulated current to the coil.

In the magnetic field irradiation step, as described above in the usage method, the positional relationship between the magnetic field generation device and a living body can be adjusted so that the living body can be irradiated with the magnetic field. The period for irradiating the living body with the magnetic field is not particularly limited as long as a useful effect, such as induction of mitophagy, on the living body can be obtained. For example, continuous irradiation for 12 hours to several months may be employed, or irradiation for only a predetermined period (for example, nighttime) may be employed.

Although Examples are presented below to specifically describe the embodiments disclosed in the present application, the Examples are merely provided for description of the embodiments. The Examples are not intended to limit or restrict the scope of the invention disclosed in the present application.

### EXAMPLES

### Example 1

### Fabrication of Magnetic Field Generation Device

### (1) Coil

A coil was fabricated by winding a copper wire having a diameter of 0.29 mm by 50 turns on an acrylic cylinder having a height of 1 cm, an inner diameter of 10 cm, and an outer diameter of 10.7 cm.

### (2) Power Supply

A power supply whose program is designed to be able to change the pulse width, the value of applying current, the frequency, the application cycle of frequencies, and the like was fabricated.

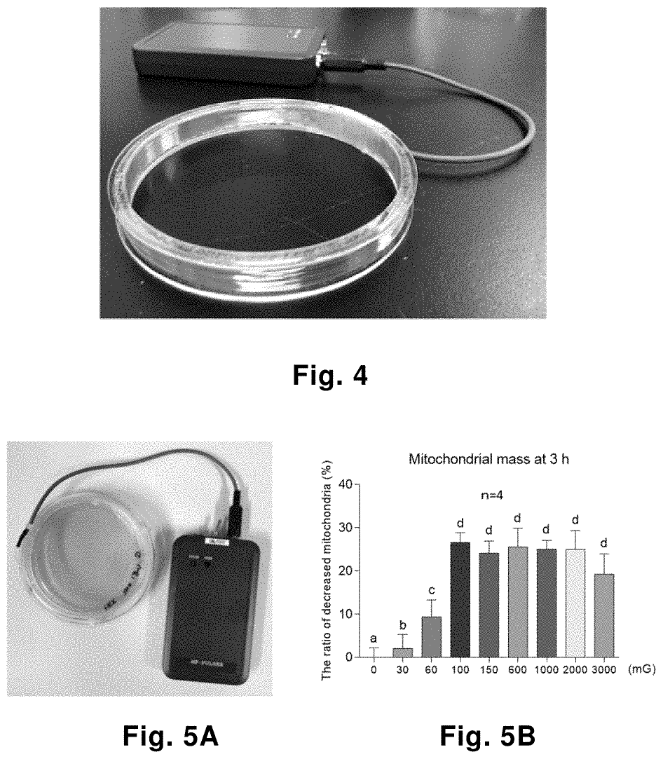

A magnetic field generation device was fabricated by electrically connecting the coil fabricated by (1) described above to the power supply fabricated by (2) described above. FIG. 4 is a photograph of a magnetic field generation device fabricated in Example 1.

### Influence of Magnetic Field Intensity on AML12 Cell Mitochondria

### Example 2

### (1) Cell

AML12 (alpha mouse liver 12 (ATCC: CRL-2254)) that is a mouse hepatocyte cell line was used for cells. A DMEM/F-12 medium (Gibco) containing 10% fetal bovine serum (FBS, Thermo Scientific), 40 mg/ml of dexamethasone (Wako), and 5 μg/mL of insulin-transferrin-sodium selenite (Sigma) was used and cultured under a wet environment at 37° C. with 5% CO₂.

### (2) Measurement of the Mitochondrial Mass Using Flow Cytometry

(2-1) Irradiation with Magnetic Field for 3 Hours

The AML12 cultured in (1) described above was seeded in a plurality of petri dishes so as to each have substantially the same cell mass. As illustrated in FIG. 5A, the petri dish was arranged inside an acrylic cylinder of the coil of the magnetic field generation device fabricated in Example 1, and pulsed current having the following conditions was applied thereto.
Pulse width: 4 msec
Frequency: a cycle with “1 Hz for 1 sec, 2 Hz for 1 sec, 3 Hz for 1 sec, 4 Hz for 1 sec, 5 Hz for 1 sec, 6 Hz for 1 sec, 7 Hz for 1 sec, and 8 Hz for 1 sec in this order” (8 sec in total) (1 to 8 Hz/8 s)

Note that, in the application of current, the value of the applying current was adjusted so that the intensity of a generated magnetic field (the highest value or the theoretical value of the intensity measured inside the coil) was 30 mG to 3000 mG. For 0 mG to 150 mG, the magnetic field was measured by a pulsed magnetic field measuring device (by Aichi Micro Intelligent Corporation). For above 600 mG, the value of current corresponding to the intensity of the magnetic field was calculated based on a theoretical value, and the calculated value of current was applied to the coil. Note that the theoretical value was found by extrapolation from the actual measured value of magnetic fields of 0 mG to 150 mG. After the magnetic field reached a set intensity, the unit cycle of application of current at the frequency described above was repeated for 3 hours to irradiate the cells with a magnetic field.

The cells were washed with a phosphate buffered saline (PBS) after culturing for 3 hours under a magnetic field environment. The mitochondrial mass was measured by:

(1) adding 50 nM of MitoTracker Green (dissolved with Thermo Scientific, M7514, Hank's equilibrium salt solution) to the cells so that the cells takes it in under a wet environment at 37° C. with 5% CO₂ for 30 minutes; and
(2) then washing the cells with the PBS, separating the cells from the petri dish by using trypsin, and measuring fluorescence per cell by using flow cytometry BD FACS Calibur (BD Biosciences).

FIG. 5B is a graph illustrating the decreased mitochondrial mass per cell after irradiating the AML12 cells with a magnetic field for 3 hours. As illustrated in FIG. 5B, after irradiation with a magnetic field of 60 mG, the mitochondrial mass per cell decreased by about 10%. Further, after irradiation with a magnetic field of 100 mG to 3000 mG, the mitochondrial mass per cell decreased by about 20% or more, and the mitochondrial mass per cell decreased by about 28% at 100 mG.

(2-2) Irradiation with Magnetic Field for 12 Hours

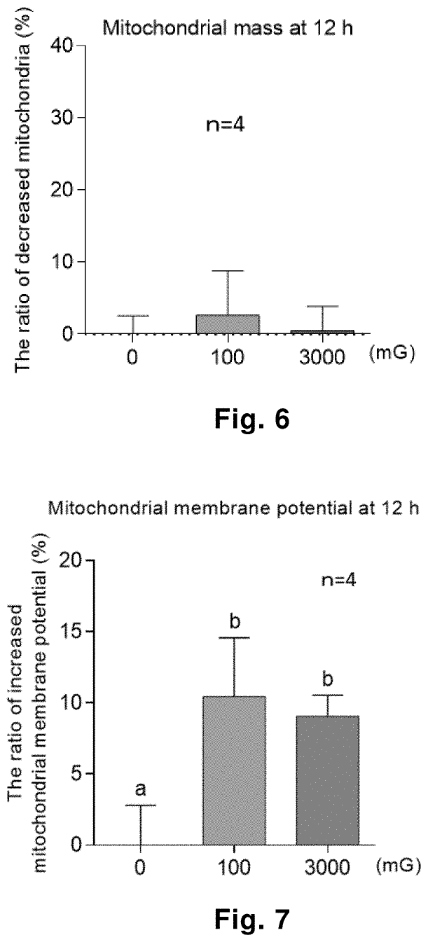

Next, a magnetic field was irradiated and fluorescence per cell was measured by the same procedure as (2-1) described above except that the magnetic field irradiation time was 12 hours. FIG. 6 is a graph illustrating the decreased mitochondrial mass per cell after irradiating the AML12 cell with a magnetic field for 12 hours (in comparison to the magnetic field irradiation time of 0 hour). As illustrated in FIG. 6, after irradiation with a magnetic field of 100 mG and 3000 mG for 12 hours, the mitochondrial mass was recovered to substantially the same as (slightly less than) the mass before the irradiation with the magnetic field. From the results illustrated in FIG. 5B and FIG. 6, it was confirmed that the mitochondrial mass per cell is once reduced and then recovered by irradiation with a magnetic field.

### (3) Measurement of the Mitochondrial Membrane Potential Using Flow Cytometry

AML12 cells were irradiated with a magnetic field for 12 hours and the mitochondrial membrane potential per cell was measured by flow cytometry by the same procedure as (2-2) described above except that, instead of MitoTracker Green, 200 nM of Tetramethylrhodamine (TMRM: Thermo Scientific, T668, dissolved in a culture medium) was used.

FIG. 7 is a graph illustrating the increased mitochondrial membrane potential per cell (compared to the magnetic field irradiation time being 0) after the AML12 cells were irradiated with a magnetic field for 12 hours. As illustrated in FIG. 7, with irradiation of the AML12 cells with the magnetic field for 12 hours, the mitochondrial membrane potential per cell increased by about 10% for both the cases of the magnetic field intensity of 100 mG and 3000 mG. In contrast, as illustrated in FIG. 6, the mitochondrial mass per cell after the AML12 cells were irradiated with the magnetic field for 12 hours was substantially the same as (slightly less than) the mass when the irradiation time was 0 hour for both the cases of the magnetic field intensity of 100 mG and 3000 mG. The mitochondrial membrane potential is an index indicating the activity of the mitochondrial electron transfer system. Therefore, from the results of FIG. 6 and FIG. 7, it was confirmed that, with irradiation of the AML12 cells with a magnetic field, the mitochondrial electron transfer system is activated, in other words, the quality of mitochondria is improved. Note that, while FIG. 6 and FIG. 7 illustrate examples in which the magnetic field intensity is 100 mG and 3000 mG, it is clear that similar results will be exhibited for other magnetic field intensities in view of the result of FIG. 5B.

### Influence of Magnetic Field Intensity on Cell with Mutation on Gene Encoding ATP Produce Protein of Mitochondria

### Example 3

### (1) Cell

Cybrid cells (transmitochondrial cybrids) were used that are hybrid cells produced by fusing human osteosarcoma cells from which mitochondria were removed and mitochondria where patient-derived mitochondria DNA was mutated.

### a: NARP3-1 Cybrid

Mitochondria DNA including 98% at mt8993T to mt8993G mutation

### b: NARP3-2 Cybrid

Mitochondria DNA including 60% at mt8993T to mt8993G mutation

The NARP3-1 and NTRP3-2 cybrids are model cells of Leigh's encephalopathy or syndrome, neurogenic muscle weakness, ataxia, and retinitis pigmentosa (NARP), respectively. Both the cells were cultured under a wet environment at 37° C. with 5% CO₂ by using a DMEM medium (Wako) containing 10% fetal bovine serum (FBS, Thermo Scientific), 1 mM of sodium pyruvate (Wako), and 0.4 mM of uridine (Sigma).

Note that, for a production procedure for the NARP3-1 cybrid and the NARP3-2 cybrid, “M. Tanaka et. al., “Gene Therapy for Mitochondrial Disease by Delivering Restriction Endonuclease Smal into Mitochondria”, J Biomed Sci 2002; 9: 534-541” can be referenced.

### (2) Experimental Method of Measuring Mitochondrial Activity of NARP Cybrid

A flux analyzer, Seahorse XFp Extracellular Flux Analyzer (Agilent Technologies), was used to measure the amount of oxygen consumption in cells in a semi-enclosed space and thereby examine the change in the mitochondrial activity due to irradiation with a magnetic field. The experiment procedure is illustrated as follows.
(a) 10,000 cells were seeded per 3.8-mm well of Seahorse XFp Cell Culture Miniplate (Agilent Technologies) and cultured under a wet environment at 37° C. with 5% CO₂ under irradiation for 9 hours. Note that the magnetic field irradiation conditions were as below:
Pulse width: 4 msec
Frequency: a cycle with “1 Hz for 1 sec, 2 Hz for 1 sec, 3 Hz for 1 sec, 4 Hz for 1 sec, 5 Hz for 1 sec, 6 Hz for 1 sec, 7 Hz for 1 sec, and 8 Hz for 1 sec in this order” (8 sec in total)
Magnetic field intensity: 100 mG
(b) On the next day, the medium was replaced with Seahorse XF Base Medium (Agilent Technologies), which was placed under a wet environment at 37° C. for 1 hour without CO₂, and the amount of oxygen consumption was then measured in accordance with the recommended protocol of a Mito stress kit for XFp (Agilent Technologies, model: 103010-100). After a base line was measured, oligomycin (ATP synthase inhibitor), FCCP (mitochondrial un-conjugating agent), rotenone (mitochondrial complex I inhibitor), antimycin (mitochondrial complex III inhibitor) were sequentially added to estimate ATP production, proton leakage, the maximum respiratory capacity, and the amount of mitochondria-independent oxygen consumption. Further, after completion of a seahorse experiment, the cells were collected by using trypsin, and the number of cells was counted and measured by TC20 automated cell counter (BioRad) to correct the amount of oxygen consumption.

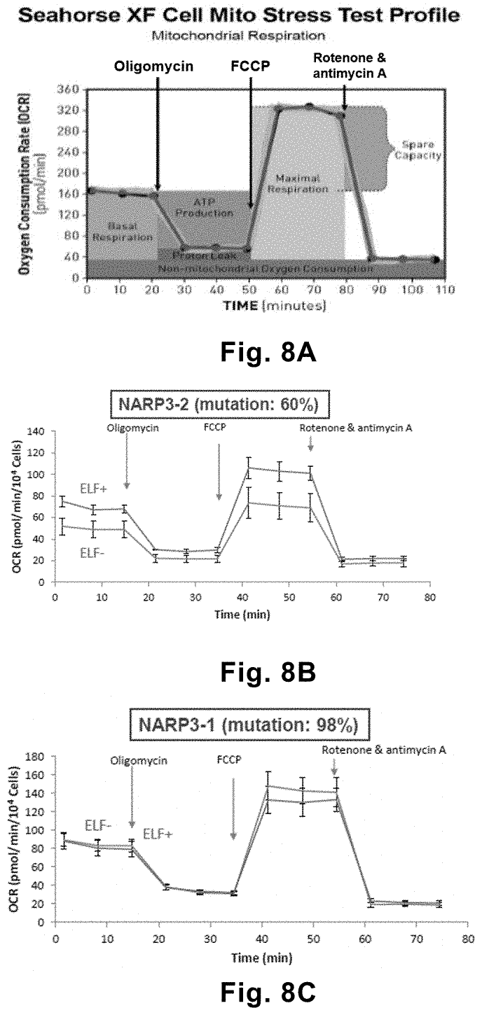

FIG. 8A illustrates a profile when the Mito stress kit was used (a change in the amount of oxygen consumption when each reagent was administered). Further, FIG. 8B is a graph illustrating changes in the amount of oxygen consumption when the NARP3-2 cybrids were irradiated with a magnetic field (ELF+ in the graph) and when the NARP3-2 cybrids were not irradiated with a magnetic field (ELF− in the graph). FIG. 8C is a graph illustrating changes in the amount of oxygen consumption when the NARP3-1 cybrids were irradiated with a magnetic field (ELF+ in the graph) and when the NARP3-1 cybrids were not irradiated with a magnetic field (ELF− in the graph).

From FIG. 8B and FIG. 8C, the following points are apparent.
(i) In the NARP3-1 in which 98% of genes encoding the mitochondrial ATP production protein has been mutated in NARP cells derived from a mitochondrial disease patient, the amount of oxygen consumption was not changed even with irradiation of the cells with a magnetic field. That is, even when the cells that have lost almost all the function of the ATP production protein were irradiated with a magnetic field, no improvement in the mitochondrial function was found.
(ii) In contrast, in the NARP3-2 in which 60% of genes encoding the mitochondrial ATP production protein has been mutated, the amount of oxygen consumption increased by irradiation of the cells with a magnetic field. That is, it was revealed that irradiation with a magnetic field improved the function of about 40% of the ATP production proteins that were unmutated.

From the results indicated by FIG. 5 to FIG. 8, it was confirmed that, with irradiation of cells with a magnetic field, mitochondrial mitophagy is first induced, thereby mitochondria of poor quality are removed (the mitochondrial mass is reduced), and mitochondria of good quality that have not been removed are then activated.

### Influence of Applied Current on Mitophagy

### Example 4

In the condition where the magnetic field intensity was 100 mG of <Example 2> (2-1) described above, an experiment to add cycles of the following frequency conditions was conducted.
1 to 2 Hz/2 s: a cycle with “1 Hz for 1 sec and 2 Hz for 1 sec in this order”
1 to 4 Hz/4 s: a cycle with “1 Hz for 1 sec, 2 Hz for 1 sec, 3 Hz for 1 sec, and 4 Hz for 1 sec in this order”
Reverse: a cycle with “8 Hz for 1 sec, 7 Hz for 1 sec, 6 Hz for 1 sec, 5 Hz for 1 sec, 4 Hz for 1 sec, 3 Hz for 1 sec, 2 Hz for 1 sec, and 1 Hz for 1 sec in this order” (in contrast to 1 to 8 Hz/8 s where the frequency is increased stepwise, the frequency is reduced stepwise)
2, 4, 6, 8 Hz/8 s: a cycle with “2 Hz for 2 sec, 4 Hz for 2 sec, 6 Hz for 2 sec, and 8 Hz for 2 sec in this order”
6 Hz: a cycle with “6 Hz”

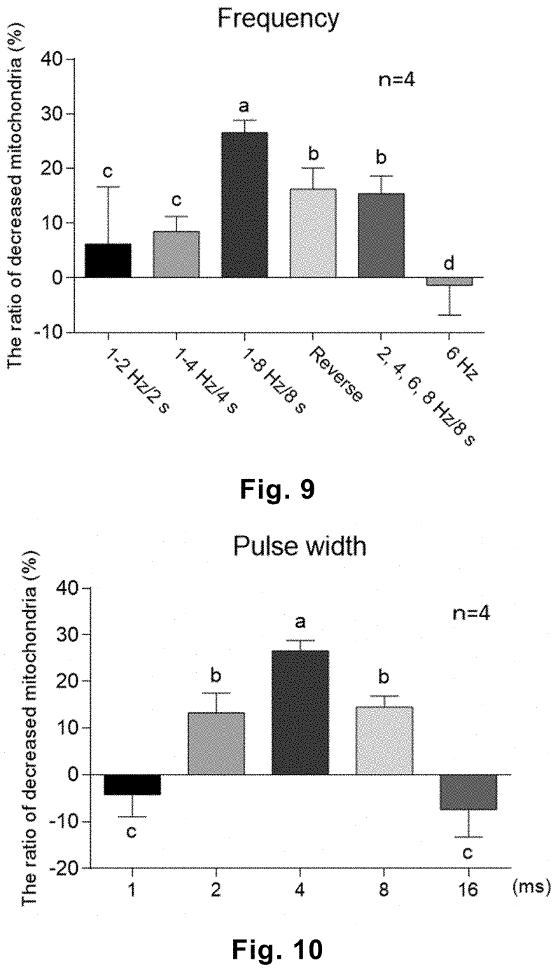

FIG. 9 illustrates the results of the experiments. As is clear from FIG. 9, when the frequency-modulated current was applied to the coil, the mitochondrial mass decreased (mitophagy was induced). In contrast, when current at constant 6 Hz was continued to be applied without a change of the frequency, no decrease in the mitochondrial mass was found. When the frequency of current applied to the coil is modulated, the frequency of a generated magnetic field is modulated in accordance with the frequency modulation of the current. It was therefore confirmed that, to induce mitophagy, it is required to modulate the frequency of a magnetic field that irradiates cells.

### Example 5

In the condition where the magnetic field intensity was 100 mG of <Example 2> (2-1) described above, additional experiments in which the pulse width was changed to 1 msec, 2 msec, 8 msec, and 16 msec in addition to 4 msec were conducted. Note that the interval time of applied current of 0 A is “⅛−w”. FIG. 10 illustrates the results of the experiments. As is clear from FIG. 10, it was revealed that a pulse width that is too short or too long is not preferable, and the pulse width is required to be adjusted as appropriate. Further, it was confirmed that mitophagy is induced when the pulse width is at least between 2 msec and 8 msec.

### Influence of Magnetic Field Irradiation on Various Cells

### Example 6

### (1) Cell

(1-1)
C2C12 (ATCC: CRL-3419): mouse striated muscle cell
Neuro2a (ATCC: CCL-131): mouse derived neuroblastoma cell
HEK293 (ATCC: CRL-1573): human fetal kidney cell
HeLa (ATCC: CCL-2): human cervical cancer cell

The above cells were cultured under a wet environment at 37° C. with 5% CO₂ by using a DMEM medium (Gibco) containing 10% fetal bovine serum (FBS, Thermo Scientific).

(1-2)

The human iPS cell (hiPS: 454-E2-FF-MD1) was cultured under a wet environment at 37° C. with 5% CO₂ by using a stemfit medium (Ajinomoto).

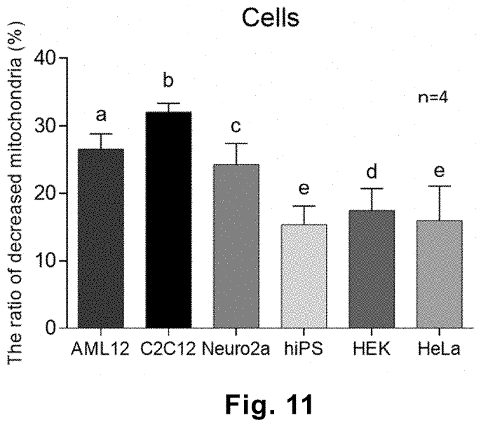

(2) In the condition where the magnetic field intensity was 100 mG of <Example 2> (2-1) described above, experiments were conducted by using different types of cells described above in addition to AML12. FIG. 11 illustrates the results of the experiments. As is clear from FIG. 11, it was confirmed that, in various types of cells containing mitochondria, irradiation with a magnetic field reduces the mitochondrial mass, in other words, induces mitophagy. Therefore, the magnetic field generation device disclosed in the present application can maintain mitochondria of good quality and thus is useful for maintaining or promoting good health or the like of a living body containing mitochondria in addition to being useful for treatment of mitochondria-related diseases.

### Influence of Magnetic Field Irradiation on Mouse

### Example 7

### (1) Parkinson's Disease Model Mouse (ASO Mouse)

Thy1l-α-Syn overexpression (ASO) mice are mice caused to excessively express human α-Syn and are used as Parkinson's disease model mice. The ASO mice were created by using C57BL/6 mice and in accordance with the procedure described in the following paper: E. Rockenstein et al., “Differential Neuropathological Alterations in Transgenic Mice Expressing a-synuclein From The Platelet-derived Growth Factor and Thy-1 Promoters”, J Neurosci Res 2002; 68: 568-578.

### (2) Exercise Test

Two magnetic field generation devices fabricated in Example 1 were arranged under a mouse-keeping cage so that the generated magnetic field was directed upward. In the cage to which the magnetic field generation devices are arranged, 8-week-old ASO mice are put therein, a magnetic field was irradiated continuously for 4 weeks, and two exercise tests were then conducted. Note that the irradiation conditions of the magnetic field were as follows.
Pulse width: 4 msec
Frequency: a cycle with “1 Hz for 1 sec, 2 Hz for 1 sec, 3 Hz for 1 sec, 4 Hz for 1 sec, 5 Hz for 1 sec, 6 Hz for 1 sec, 7 Hz for 1 sec, and 8 Hz for 1 sec in this order” (8 sec in total) (1 to 8 Hz/8 s)
Magnetic field intensity: 100 mG

Further, C57BL/6 wild type mice (WT) and ASO mice that have not been irradiated with a magnetic field were used for control groups.

### (2-1) Rota Rod Test

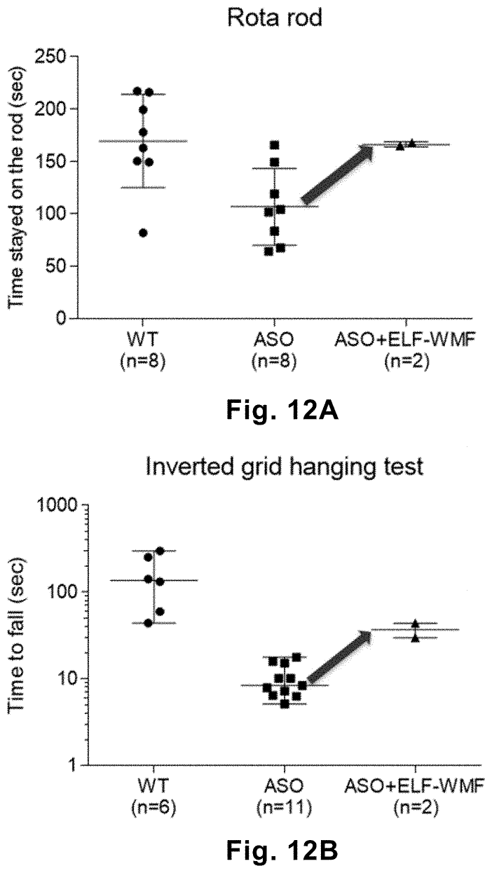

A Rota rod (Ugo Basile, Comerio, Italy) was used, a mouse was placed on a rotating rod of this device, and the time until the mouse fell from the rotating rod was measured. Note that the same test was made for the purpose of training of mice on the day before the experiment. In the experiment, the Rota rod test was conducted under the condition where the rotational rate of the rod was gradually accelerated to 4 to 40 rpm over 240 sec, and the time during which the mouse was able to continue to stay on the rod was measured. When the mouse did not fall, the time during which the mouse was able to continue to stay on the rod was determined as 240 sec. The test was conducted for three times with a one-hour break after each measurement. FIG. 12A indicates the result.

### (2-2) Inverted Grid Hanging Test

A mouse was place at the center of a metal mesh of 50 cm by 50 cm with a mesh width of 1 cm. The metal mesh was then inverted to cause the mouse to hang from the metal mesh and was then fixed at 50 cm above a cushion. The time until the mouse fell from the metal mesh was recorded. The time limit was 5 minutes. FIG. 12B indicates the result.

First, as is clear from FIG. 12A, the time that the mice were able to stay on the rotating rod was shorter in the Parkinson's model mice (ASO) group than in the wild type (WT) group. However, the time that the mice were able to stay on the rotating rod increased in the group of Parkinson's model mice (ASO) that were irradiated with a magnetic field for 4 weeks (ASO+ELF−WMF).

Further, as is clear from FIG. 12B, the time that the mice were able to hang from the metal mesh was shorter in the Parkinson's model mice (ASO) group than in the wild type (WT) group. However, the time that the mice were able to hang from the metal mesh increased in the group of Parkinson's model mice (ASO) that were irradiated with a magnetic field for 4 weeks (ASO+ELF−WMF).

From the results set forth, it was confirmed that the magnetic field generation device disclosed in the present application can be used for treatment of Parkinson's disease.

### Example 8

### (1) Creation of Depression Model Mouse

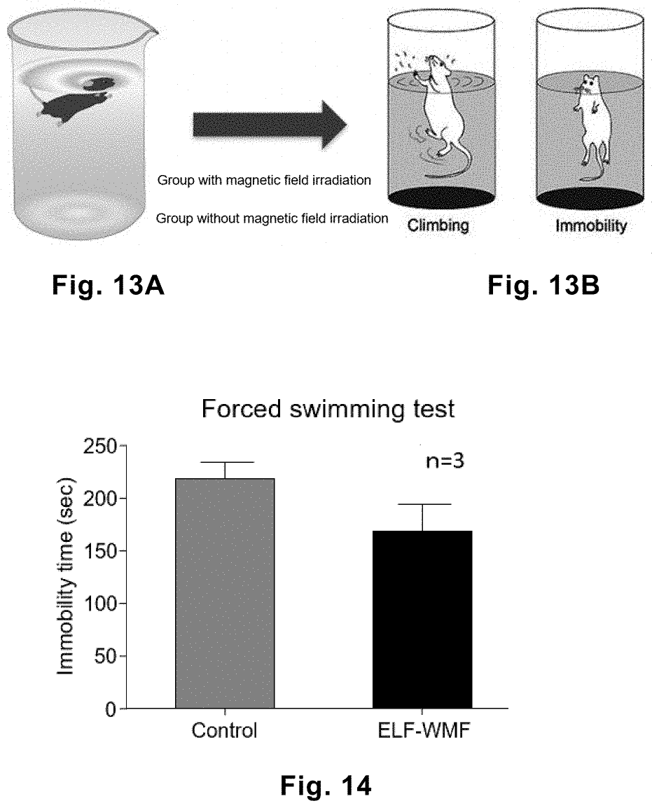

A method of creating depression model mice will be described with reference to FIG. 13. ICR mice (10 weeks old) purchased from CLEA Japan, Inc. (Tokyo, Japan) were used for this creation. A cylinder with a diameter of 10 cm was filled with water at 25° C. including 0.1% surfactant Clean Ace S (AsOne) (to a depth where the mouse's feet does not touch the bottom, about 1000 mL), and the ICR mice were forced to swim therein for 15 minutes to create depression model mice (FIG. 13A).

### (2) Swimming Test

Next, the procedure of a swimming experiment for the depression model mice will be described with reference to FIG. 13. The depression model mice created by (1) described above were returned to the same cage as described in Example 7, and a magnetic field irradiation was performed for 24 hours under the same conditions as those in Example 7 (group with magnetic field irradiation). Note that a group without magnetic field irradiation in which no magnetic field irradiation was applied to the depression model mice was used as a control group. The second forced swimming was then applied in water at 25° C. (containing no surfactant) for 6 minutes, and the immobility time during the latter 4 minutes was measured. In FIG. 13B, while “Climbing” represents normal escaping behavior, “Immobility” represents a state of giving up escaping behavior (depression state).

FIG. 14 indicates the results of the experiments. As is clear from FIG. 14, the immobility time of the group with magnetic field irradiation (ELF-WMF) was shorter than that of the group without magnetic field irradiation (Control).

From the results set forth, it was confirmed that the magnetic field generation device disclosed in the present application can be used for treatment of depression.

### INDUSTRIAL APPLICABILITY

The magnetic field generation device and the magnetic field irradiation method disclosed in the present application can induce mitophagy to improve mitochondrial activity. Further, the magnetic field generation device and the magnetic field irradiation method disclosed in the present application are useful for treating mitochondria-related diseases such as Parkinson's disease, depression, or the like and maintaining or promoting good health or the like of a living body containing mitochondria. Therefore, the magnetic field generation device and the magnetic field irradiation method disclosed in the present application are useful for industries for manufacturing medical devices or the like.

### LIST OF REFERENCE NUMERALS

1 magnetic field generation device
2 coil
21 support
3 power supply

1. A magnetic field generation device comprising:
a coil; and
a power supply,
wherein the power supply is configured to apply pulsed and frequency-modulated current to the coil, and
wherein the maximum value of a generated magnetic field is 60 mG to 3000 mG.

2. The magnetic field generation device according to claim 1, wherein a pulse width of the current is selected from 2 to 8 msec.

3. The magnetic field generation device according to claim 1, wherein the power supply is configured to repeatedly apply
a cycle in which the frequency increases during a predetermined period, or
a cycle in which the frequency decreases during a predetermined period
to the coil.

4. The magnetic field generation device according to claim 3,
wherein the frequency is the number of pulses applied to the coil per second, and
wherein during the predetermined period,
the frequency increases stepwise within a range selected from 1 Hz to 8 Hz, or
the frequency decreases stepwise within a range selected from 8 Hz to 1 Hz.

5. The magnetic field generation device according to claim 4, wherein the predetermined period is selected from 2 sec to 8 sec.

6. The magnetic field generation device according to claim 1, wherein the magnetic field generation device is used for treatment of a mitochondria-related disease.

7. A magnetic field irradiation method for a living body by using a magnetic field generation device including a coil and a power supply, the magnetic field irradiation method comprising:
a magnetic field irradiation step of irradiating a living body with a magnetic field having the maximum value of 60 mG to 3000 mG generated by the magnetic field generation device,
wherein in the magnetic field irradiation step, the power supply applies pulsed and frequency-modulated current to the coil.

8. The magnetic field irradiation method according to claim 7, wherein a pulse width of the current is selected from 2 to 8 msec.

9. The magnetic field irradiation method according to claim 7, wherein the power supply is configured to repeatedly apply
a cycle in which the frequency increases during a predetermined period, or
a cycle in which the frequency decreases during a predetermined period
to the coil.

10. The magnetic field irradiation method according to claim 9,
wherein the frequency is the number of pulses applied to the coil per second, and
wherein during the predetermined period,
the frequency increases stepwise within a range selected from 1 Hz to 8 Hz, or
the frequency decreases stepwise within a range selected from 8 Hz to 1 Hz.

11. The magnetic field irradiation method according to claim 10, wherein the predetermined period is selected from 2 sec to 8 sec.

12. The magnetic field irradiation method according to claim t, wherein the magnetic field irradiation method is used for a method of treating a mitochondria-related disease.

13. The magnetic field generation device according to claim 1, wherein pulsed current is current having substantially a rectangular waveform.

14. The magnetic field generation device according to claim 2, wherein pulsed current is current having substantially a rectangular waveform.

15. The magnetic field irradiation method according to claim 7, wherein pulsed current is current having substantially a rectangular waveform.

16. The magnetic field irradiation method according to claim 8, wherein pulsed current is current having substantially a rectangular waveform.

17. The magnetic field generation device according to claim 2, wherein the power supply is configured to repeatedly apply
a cycle in which the frequency increases during a predetermined period, or
a cycle in which the frequency decreases during a predetermined period
to the coil.

18. The magnetic field generation device according to claim 13, wherein the power supply is configured to repeatedly apply
a cycle in which the frequency increases during a predetermined period, or
a cycle in which the frequency decreases during a predetermined period
to the coil.

19. The magnetic field generation device according to claim 14, wherein the power supply is configured to repeatedly apply
a cycle in which the frequency increases during a predetermined period, or
a cycle in which the frequency decreases during a predetermined period
to the coil.

20. The magnetic field generation device according to claim 17,
wherein the frequency is the number of pulses applied to the coil per second, and
wherein during the predetermined period,
the frequency increases stepwise within a range selected from 1 Hz to 8 Hz, or
the frequency decreases stepwise within a range selected from 8 Hz to 1 Hz.
